SEQUENCE LISTING (6 sequences)

SEQ ID NO: 1 - protein, 16 aa, Bacillus anthracis
HGNAEVHASFFDIGGS

SEQ ID NO: 2 - protein, 20 aa, Bacillus anthracis
QIDIRDSLSEEEKELLNRIQ

SEQ ID NO: 3 - protein, 35 aa, Bacillus anthracis
TEEKEFLKKLQIDIRDSLSEEEKELLNRIQVDSSN

SEQ ID NO: 4 - protein, 29 aa, Bacillus anthracis
LKKLQIDIRDSLSEEEKELLNRIQVDSSN

SEQ ID NO: 5 - protein, 25 aa, Bacillus anthracis
QIDIRDSLSEEEKELLNRIQVDSSN

SEQ ID NO: 6 - protein, 188 aa, Woodchuck hepatitis virus
MDIDPYKEFGSSYQLLNFLPLDFFPDLNALVDTATALYEEELTGREHCSPHHTAIRQALVCWDELTKLIAWMSSNITSEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

(19) United States
(12) Patent Application Publication
(10) Pub. No.: US 2025/0152692 A1
(43) Pub. Date: May 15, 2025

(54) IMMUNOGENS TARGETING ANTHRAX

(71) Applicants: The Regents of the University of Michigan, Ann Arbor, MI (US); The United States of America as represented by the Department of Veteran Affairs, Washington, DC (US); VLP Biotech, Inc., San Diego, CA (US)

(72) Inventors: Kemp B. Cease, Ann Arbor, MI (US); Jon Oscherwitz, Ann Arbor, MI (US); David C. Whitacre, San Diego, CA (US)

(21) Appl. No.: 18/715,507

(22) PCT Filed: Dec. 1, 2022
(86) PCT No.: PCT/US2022/080759

§ 371 Date: May 31, 2024

Related U.S. Application Data
(60) Provisional application No. 63/284,841, filed on Dec. 1, 2021.

(51) Int. Cl. A61K 39/07; A61K 39/00; A61P 37/04; C07K 14/32; C12N 15/86

(52) U.S. Cl. CPC A61K 39/07; A61P 37/04; C07K 14/32; C12N 15/86; A61K 2039/5258; C12N 2730/10023; C12N 2730/10043

(57) ABSTRACT

The present disclosure provides immunogens, immunogenic compositions, vaccines based on neutralizing epitopes from lethal factor peptide (LF) and loop neutralizing determinant (LND) of protective antigen, and methods of using thereof for treating and preventing infection and disease caused by Bacillus anthracis.

### CROSS-REFERENCE TO RELATED APPLICATIONS

This application claims the benefit of U.S. Provisional Application No. 63/284,841, filed Dec. 1, 2021, the content of which is herein incorporated by reference in its entirety.

### STATEMENT REGARDING FEDERALLY SPONSORED RESEARCH OR DEVELOPMENT

This invention was made with government support under A1106153 awarded by the National Institutes of Health. The government has certain rights in the invention.

### FIELD

The present disclosure provides immunogens, immunogenic compositions, vaccines, and methods of using thereof for treating and preventing anthrax.

### SEQUENCE LISTING STATEMENT

The contents of the electronic sequence listing titled UM-38078.601.xml (Size: 6,456 bytes; and Date of Creation: Nov. 23, 2022) is herein incorporated by reference in its entirety.

### BACKGROUND

Anthrax is a serious infection caused by the spore-forming bacterium Bacillus anthracis. The infectivity of B. anthracis can be attributed to two factors: the polyglutamic acid capsule and the anthrax toxin. Anthrax toxin consists of the three distinct polypeptides known as protective antigen (PA), oedema factor (EF), and lethal factor (LF). The toxin components act in specific binary combinations of PA and EF to form oedema toxin (ET), which causes tissue oedema, and of PA and LF to form lethal toxin (LT), which causes lysis of monocyte and macrophage cells. Lethal toxin is considered to be the principal cause of anthrax-associated death as a consequence of its cytotoxic effects on peripheral macrophages and other cells.

Anthrax Vaccine Adsorbed (AVA, BioThrax®), the currently licensed anthrax vaccine in the U.S., confers a high degree of protection from inhalation spore challenge in rabbits and non-human primates. However, there are concerns about the use of Bio Thrax as the primary vaccine against anthrax due to a number of factors, including: lack of development of protective levels of neutralizing antibody, lack of the antibody to neutralize the toxin, a cumbersome immunization protocol, frozen storage requirements, and incidence of reactogenicity.

### SUMMARY

Disclosed herein are immunogenic compositions comprising at least one or each of: an antigenic Bacillus anthracis lethal factor peptide and an antigenic Bacillus anthracis protective antigen peptide, wherein when the lethal factor peptide is absent the protective antigen peptide is inserted within an amino acid sequence of a polypeptide capable of forming a virus like particle.

In some embodiments, the antigenic anthrax protective antigen peptide comprises the amino acid sequence HGNAEVHASFFDIGGS (SEQ ID NO: 1).

In some embodiments, the antigenic anthrax lethal factor peptide comprises the amino acid sequence QIDIRDSLSEEEKELLNRIQ (SEQ ID NO: 2). In some embodiments, the antigenic anthrax lethal factor peptide is selected from the group consisting of the amino acid sequences of SEQ ID NO:2, TEEKEFLKKLQIDIRDSLSEEEKELLNRIQVDSSN (SEQ ID NO:3), LKKLQIDIRDSLSEEEKELLNRIQVDSSN (SEQ ID NO:4), and QIDIRDSLSEEEKELLNRIQVDSSN (SEQ ID NO:5).

In some embodiments, the lethal factor peptide is inserted within an amino acid sequence of a polypeptide capable of forming a virus like particle.

In some embodiments, the lethal factor peptide is present and the protective antigen peptide is inserted within an amino acid sequence of a polypeptide capable of forming a virus like particle.

In some embodiments, the lethal factor peptide and the protective antigen peptide are each individually inserted within an amino acid sequence of a polypeptide capable of forming a virus like particle. In some embodiments, the lethal factor peptide and the protective antigen peptide are in distinct virus like particles.

In some embodiments, the polypeptide capable of forming a virus like particle is a woodchuck hepatitis DNA virus core antigen. In some embodiments, the amino acid sequence of the woodchuck hepatitis DNA virus core antigen comprises an amino acid sequence having at least 70% identity (e.g., at 70%, 75%, 80%, 85%, 90%, 95%, 98%, or 99% identity) to SEQ ID NO: 6. In some embodiments, insertion in the amino acid sequence of the woodchuck hepatitis DNA virus core antigen is at position 78 of SEQ ID NO: 6.

Also disclosed herein are vaccines comprising the immunogenic compositions disclosed herein.

Further provided are methods for reducing or preventing anthrax infection in a subject in need thereof comprising administering to the subject an effective amount of the immunogenic compositions or vaccines disclosed herein. In some embodiments, the administering comprises an initial immunization and at least one subsequent immunization. In some embodiments, the administering comprises a single immunization.

Additionally disclosed are nucleic acids encoding antigenic Bacillus anthracis lethal factor peptide, encoding antigenic Bacillus anthracis lethal factor peptide inserted within an amino acid sequence of a polypeptide capable of forming a virus like particle, and antigenic Bacillus anthracis protective antigen peptide inserted within an amino acid sequence of a polypeptide capable of forming a virus like particle.

In some embodiments, the antigenic anthrax protective antigen peptide comprises the amino acid sequence HGNAEVHASFFDIGGS (SEQ ID NO: 1).

In some embodiments, the antigenic anthrax lethal factor peptide comprises the amino acid sequence QIDIRDSLSEEEKELLNRIQ (SEQ ID NO: 2). In some embodiments, the lethal factor peptide is selected from the group consisting of the amino acid sequences of SEQ ID NO:2, TEEKEFLKKLQIDIRDSLSEEEKELLNRIQVDSSN (SEQ ID NO:3), LKKLQIDIRDSLSEEEKELLNRIQVDSSN (SEQ ID NO:4), and QIDIRDSLSEEEKELLNRIQVDSSN (SEQ ID NO:5).

In some embodiments, the antigenic anthrax protective antigen peptide comprises the amino acid sequence HGNAEVHASFFDIGGS (SEQ ID NO: 1).

In some embodiments, the polypeptide capable of forming a virus like particle is a woodchuck hepatitis DNA virus core antigen. In some embodiments, the amino acid sequence of the woodchuck hepatitis DNA virus core antigen comprises an amino acid sequence having at least 70% identity (e.g., at 70%, 75%, 80%, 85%, 90%, 95%, 98%, or 99% identity) to SEQ ID NO: 6. In some embodiments, insertion in the amino acid sequence of the woodchuck hepatitis DNA virus core antigen is at position 78 of SEQ ID NO: 6.

Other aspects and embodiments of the disclosure will be apparent in light of the following detailed description and accompanying figures.

### BRIEF DESCRIPTION OF THE DRAWINGS

FIG. 1 is a protein structural model for monomeric B. anthracis protective antigen (PA) (PA63) based on PDB 1TZN with the loop neutralizing determinant (LND) indicated with its respective sequence (HGNAEVHASFFDIGGS; SEQ ID NO: 1) shown at the bottom.

FIG. 2 is a protein structural model of the anthrax PA pore model. The LND appears at the bottom beta-hairpin turn. One of the seven occurrences of the LND in the pore model is shown in red.

FIG. 3 is an image based on the protein structural model of the Woodchuck Hepatitis B core capsid protein monomer (amino acids 1-187) depicting the major immunodominant loop region with the labeled epitope insertion sites, and the C-terminal domain which binds bacterial RNA and acts as a toll receptor 7/8 ligand. The hepatitis B core protein monomer self-assembles into icosahedral nanoparticles comprising 240 monomers (shown at left) based on electron microscopic analysis. Tube structures represent sequences which form alpha-helices.

FIG. 4 is the transmission electron microscopy (TEM) micrographs at 40,000× of the non-lyophilized LND-VLP (1) and the lyophilized and resolubilized LND-VLP (ii) with their respective dynamic light scattering (DLS) graphs shown in iii and iv. Mean particles diameters were 45.70 nm for the non-lyophilized LND-VLP and 45.65 nm for the lyophilized and resolubilized LND-VLP.

FIG. 5 is a graph of the serum neutralization titers from sera obtained at 8 weeks for groups of rabbits (n=3) immunized twice at 0 and 5 weeks with either the VLP66 or the VLP66-LYO using Alhydrogel/MPLA adjuvant. Titers are expressed as NF50s and horizontal lines represent geometric means. Shown for comparative purpose is the neutralization for a recombinant PA hyperimmune rabbit control serum as described in methods.

FIG. 6 is a protein model of Lethal Factor 3D structure (PDB IJKY). The neutralizing epitope is indicated with the corresponding amino acid sequence (QIDIRDSLSEEEKELLNRIQ, SEQ ID NO: 2) shown at the bottom.

FIG. 7 is a graph of the serum neutralization titers from sera obtained at 8 weeks for groups of rabbits (n=3) immunized twice at 0 and 5 weeks with VLPs displaying putative LF neutralizing epitope sequences mixed with Alhydrogel/MPLA adjuvant. Titers are expressed as NF50s and horizontal lines represent geometrie means.

FIG. 8 is the TEM micrographs at 40,000× of the non-lyophilized LF-VLP (1) and the lyophilized and resolubilized LF-VLP (11) with their respective dynamic light scattering (DLS) graphs shown in iii and iv. Mean particles diameters were 54.64 nm for the non-lyophilized LF-VLP and 53.98 nm for the lyophilized and resolubilized LF-VLP.

FIG. 9 is a graph of the serum neutralization titers from sera obtained at 8 weeks for groups of rabbits (n=2) immunized twice at 0 and 5 weeks with the VLP148LE or the VLP148LE-LYO mixed with Alhydrogel/MPLA. Shown for comparative purpose is the neutralization for a recombinant PA hyperimmune rabbit control serum as described in methods and AVR801. Titers are expressed as NF50s and horizontal lines represent geometric means.

FIG. 10 is a graph of the serum neutralization titers from sera obtained at 8 weeks for a group of rabbits (n=8) immunized twice at 0 and 5 weeks with a mixture of the LND-VLP and the LF-VLP mixed with Alhydrogel/MPLA, and a second group of rabbits (n=10) immunized twice at day 0 and 28 with recombinant PA83 mixed with Alhydrogel. Titers are expressed as NF50s and horizontal lines represent geometric means. For the LND-VLP/LF-VLP group the GMT is 12.81, while the recombinant PA group is 13.12.

FIG. 11 is a graph of the serum neutralization titers in 8 week sera for a group of rabbits (n=8) immunized twice at 0 and 5 weeks with a mixture of the LND-VLP and the LF-VLP mixed with Alhydrogel/MPLA. Prior to assessment in the TNA, sera were pre-incubated with either media alone, irrelevant VLP, LF-VLP or both the LND-VLP and the LF-VLP. Titers are expressed as NFS0s and horizontal lines represent geometrie means.

FIG. 12 is a graph of the serum neutralization titers in sera obtained at 3, 4, 5 and 6 weeks from a group of rabbits (n=6) immunized one time with a mixture of the LND-VLP and the LF-VLP in an emulsion with Incomplete Freund's adjuvant. Titers are expressed as NF50s and horizontal lines represent geometric means. The NFS0 GMT is 0.15 at 3 weeks, 0.23 at 4 weeks, 1.02 at 5 weeks and 3.01 at 6 weeks.

### DETAILED DESCRIPTION

The present disclosure provides anthrax immunogens, immunogenic compositions, and vaccines based on a neutralizing determinant from Bacillus anthracis lethal factor (LF). The immunogens, immunogenic compositions, and vaccines target a short segment of protein from anthrax LF, a critical component of lethal toxin (LeTx). The levels of LeTx-neutralizing antibody elicited by this vaccine immunogens, immunogenic compositions, and vaccines exceed those shown to be sufficient for protection of rabbits from inhalation anthrax resulting from a high-dose experimental challenge with aerosolized Ames strain anthrax spores. The immunogen may be incorporated into a virus like particle which displays the target epitope in the major immunodominant region.

There is minimal to no LF in the currently licensed anthrax vaccine and LF-neutralizing specificity is absent from the repertoire of antibodies elicited by the vaccine. Consequently, the disclosed immunogens, immunogenic compositions, and vaccines, which perform equally well when combined with other vaccines, may compliment the protection achievable with current vaccines thereby increasing reliability of protection. Importantly, the disclosed immunogens, immunogenic compositions, and vaccines may provide immune protection from inhalation anthrax in circumstances where the attack strain produces LeTx that escapes immunity elicited by Biothrax or conferred by passive administration of current anti-PA antibodies, representing a potential countermeasure for even the most highly engineered strains. Additionally, the disclosed immunogens, immunogenic compositions, and vaccines can be lyophilized (freeze-dried) and stored with no loss of activity upon reconstitution in water and use in vaccination.

Section headings as used in this section and the entire disclosure herein are merely for organizational purposes and are not intended to be limiting.

### 1. Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. As used herein, comprising a certain sequence or a certain SEQ ID NO usually implies that at least one copy of said sequence is present in recited peptide or polynucleotide. However, two or more copies are also contemplated. The singular forms “a.” “and” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms sball include pluralities and plural terms shall include the singular.

The terms “immunogen” and “immunogenic composition,” as used herein, refer to a molecule or composition which contains one or more epitopes that will stimulate the immune response in a host organism to generate a cellular immunogen-specific immune response, and/or a humoral antibody response.

The term “immunization,” as used herein, refers to a process that increases an organisms' reaction to an antigen and thereby improves its ability to resist or overcome infection.

“Polynucleotide” or “oligonucleotide” or “nucleic acid,” as used herein, means at least two nucleotides covalently linked together. The polynucleotide may be DNA, both genomic and cDNA, RNA, or a hybrid, where the polynucleotide may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods. Polynucleotides may be single- or double-stranded or may contain portions of both double stranded and single stranded sequence. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The proteins may be modified by the addition of sugars, lipids or other moieties not included in the amino acid chain. The terms “polypeptide” and “protein,” are used interchangeably herein.

As used herein, the term “percent sequence identity” refers to the percentage of nucleotides or nucleotide analogs in a nucleic acid sequence, or amino acids in an amino acid sequence, that is identical with the corresponding nucleotides or amino acids in a reference sequence after aligning the two sequences and introducing gaps, if necessary, to achieve the maximum percent identity. A number of mathematical algorithms for obtaining the optimal alignment and calculating identity between two or more sequences are known and incorporated into a number of available software programs. Examples of such programs include CLUSTAL-W, T-Coffee, and ALIGN (for alignment of nucleic acid and amino acid sequences), BLAST programs (e.g., BLAST 2.1, BL2SEQ, and later versions thereof) and FASTA programs (e.g., FASTA3×, FAS™, and SSEARCH) (for sequence alignment and sequence similarity searches). Sequence alignment algorithms also are disclosed in, for example, Altschul et al., J. Molecular Biol., 215 (3): 403-410 (1990), Beigert et al., Proc. Natl. Acad. Sci. USA, 106 (10): 3770-3775 (2009), Durbin et al., eds., Biological Sequence Analysis: Probabilistic Models of Proteins and Nucleic Acids, Cambridge University Press, Cambridge, UK (2009), Soding, Bioinformatics, 21 (7): 951-960 (2005), Altschul et al., Nucleic Acids Res., 25 (17): 3389-3402 (1997), and Gusfield, Algorithms on Strings, Trees and Sequences, Cambridge University Press, Cambridge UK (1997)).

The term “vaccine,” as used herein, refers to any pharmaceutical composition containing at least one immunogen, which composition can be used to prevent or treat a disease or condition in a subject.

As used herein, the term “virus like particle” refers to a structure resembling a virus particle, but which has been demonstrated to be non-pathogenic.

As used herein, the term “preventing” refers to partially or completely delaying onset of an infection, disease, disorder and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

As used herein, “treat,” “treating,” and the like means a slowing, stopping, or reversing of progression of a disease or disorder when provided a composition described herein to an appropriate control subject. The term also means a reversing of the progression of such a disease or disorder to a point of eliminating or greatly reducing the cell proliferation. As such, “treating” means an application or administration of the compositions described herein to a subject, where the subject has a disease or a symptom of a disease, where the purpose is to cure, heal, alleviate, relieve, alter, remedy, ameliorate, improve, or affect the disease or symptoms of the disease.

A “subject” or “patient” may be human or non-human and may include, for example, animal strains or species used as “model systems” for research purposes, such a mouse model as described herein. Likewise, patient may include citber adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: bumans, non-human primates such as chimpanzees, and other apes and monkey species; farm animals such as cattle, horses, sheep, goats, swine; domestic animals such as rabbits, dogs, and cats; laboratory animals including rodents, such as rats, mice, and guinea pigs, and the like. Examples of non-mammals include, but are not limited to, birds, fish, and the like. In one embodiment of the methods and compositions provided herein, the mammal is a human.

As used herein, the terms “providing,” “administering,” and “introducing” are used interchangeably herein and refer to the placement of the compositions of the disclosure into a subject by a method or route which results in at least partial localization of the composition to a desired site. The compositions can be administered by any appropriate route which results in delivery to a desired location in the subject.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. Immunogens and Immunogenic Compositions

The present disclosure provides immunogenic compositions comprising at least one or each of an antigenic Bacillus anthracis lethal factor peptide and an antigenic Bacillus anthracis protective antigen peptide. If the lethal factor peptide is absent from the composition, the protective antigen peptide is inserted within an amino acid sequence of a polypeptide capable of forming a virus like particle. As such, the disclosure provides an immunogen comprising an antigenic Bacillus anthracis lethal factor peptide. The disclosure also provides an immunogen comprising an antigenic Bacillus anthracis protective antigen peptide inserted within an amino acid sequence of a polypeptide capable of forming a virus like particle.

In some embodiments, the protective antigen peptide comprises, consists essentially of, or consists of the amino acid sequence of SEQ ID NO: 1 (HGNAEVHASFFDIGGS) and/or a functional variant thereof. The peptide sequence may contain one or more modifications or alterations to the primary amino acid sequence such that the functional variant retains its immunostimulatory effect. The functional variant should retain greater than 50% of the activity of the original peptide. Quantitative binding and antibody binding assays may be used to readily determine functional variants of interest.

In some embodiments, the antigenic anthrax lethal factor peptide comprises, consists essentially of, or consists of the amino acid sequence of SEQ ID NO: 2 (QIDIRDSLSEEEKELLNRIQ) and/or a functional variant thereof. In certain embodiments, the lethal factor peptide is selected from the group consisting of the amino acid sequences of SEQ ID NO: 2, SEQ ID NO:3, SEQ ID NO:4, and SEQ ID NO:5 and/or functional variants thereof. The peptide sequences may contain one or more modifications or alterations to the primary amino acid sequence such that the functional variant retains its immunostimulatory effect. The functional variant should retain greater than 50% of the activity of the original peptide. Quantitative binding and antibody binding assays may be used to readily determine functional variants of interest.

The amino acid modifications or alterations can be conservative, semi-conservative, or non-conservative replacement or substitution. The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz and Schirmer, Principles of Protein Structure, Springer-Verlag, New York (1979)). According to such analyses, groups of amino acids may be defined where amino acids within a group exchange preferentially with each other, and therefore resemble each other most in their impact on the overall protein structure (Schulz and Schirmer, supra).

Examples of conservative amino acid substitutions include substitutions of amino acids within the sub-groups described above, for example, lysine for arginine and vice versa such that a positive charge may be maintained, glutamic acid for aspartic acid and vice versa such that a negative charge may be maintained, serine for threonine such that a free-OH can be maintained, and glutamine for asparagine such that a free —NH₂ can be maintained. “Semi-conservative mutations” include amino acid substitutions of amino acids within the same groups listed above, but not within the same sub-group. For example, the substitution of aspartic acid for asparagine, or asparagine for lysine, involves amino acids within the same group, but different sub-groups. “Non-conservative mutations” involve amino acid substitutions between different groups, for example, lysine for tryptophan, or phenylalanine for serine, etc.

In some embodiments, the lethal factor peptide is inserted within an amino acid sequence of a polypeptide capable of forming a virus like particle.

In some embodiments, when the lethal factor peptide is present, the protective antigen peptide is inserted within an amino acid sequence of a polypeptide capable of forming a virus like particle.

In some embodiments, at least one or both of the antigenic Bacillus anthracis lethal factor peptide and the antigenic Bacillus anthracis protective antigen peptide are inserted into an amino acid sequence of a polypeptide capable of forming a virus like particle. In exemplary embodiments, the lethal factor peptide and the protective antigen peptide are each individually inserted within an amino acid sequence of a polypeptide capable of forming a virus like particle.

When both peptides are each individually inserted in a polypeptide capable of forming a VLP, the VLP may include one of both of the antigenic peptides. Therefore, the VLPs may be distinct for each of the antigenic peptides or may comprise subunits which contain both of the antigenic peptides. In some embodiments, the lethal factor peptide and the protective antigen peptide are in distinct virus like particles, such that the composition comprises one VLP with polypeptides in which the antigenic Bacillus anthracis lethal factor peptide is inserted and a second VLP with polypeptides in which the antigenic Bacillus anthracis protective antigen peptide is inserted.

Examples of polypeptide capable of forming a virus like particle (VLP) include, but are not limited to, polypeptides of Qβ, MS2, PP7, AP205 and other bacteriophage coat proteins and the capsid and core proteins/polypeptides of a wide variety of virus families including Parvoviridae (e.g., adeno-associated virus), Retroviridae (e.g., HIV), Flaviviridae (e.g., Hepatitis C virus), Hepadnaviridae (e.g., Hepatitis B virus), Paramyxoviridae (e.g., measles virus), Paramyxoviridae (e.g., Nipah), Togaviridae (e.g., Sindbis virus), Picornaviridae (e.g., foot-and-mouth disease virus), Caliciviridae (e.g., Nowalk virus), Bromoviridae (e.g., cowpea mosaic virus), polyomaviridae (e.g., JC virus), and Papillomaviridae (HPV).

The VLPs can be synthesized chemically or through a biological process, include a variety of cell culture systems, including bacterial, mammalian, insect, yeast, and plant cells. In certain embodiments, the VLP can comprise recombinant polypeptides of any of the virus known to form a VLP to create a recombinant VLP. The virus-like particle can further comprise, or alternatively consist of, one or more fragments of such polypeptides, as well as variants of such polypeptides. Variants of polypeptides can share, for example, at least 80%, 85%, 90%, 95%, 97%, or 99% identity at the amino acid level with their wild-type counterparts.

In some embodiments, the polypeptide capable of forming a virus like particle is a woodchuck hepatitis DNA virus core antigen. The basic subunit of the core particle is a 21 kDa polypeptide monomer that spontaneously assembles into a 240-subunit structure of about 34 nm in diameter.

Any of the known hepatitis virus core proteins sequences may be appropriate for use with peptide described herein, such as those described in U.S. Pat. No. 7,883,843, incorporated herein in its entirety by reference. In some embodiments, the hepatitis virus is woodchuck hepatitis DNA virus. In some embodiments, the amino acid sequence of the woodchuck hepatitis DNA virus core antigen may comprise an amino acid sequence having at least 70% identity (e.g., at 70%, 75%, 80%, 85%, 90%, 95%, 98%, or 99% identity) to SEQ ID NO: 6.

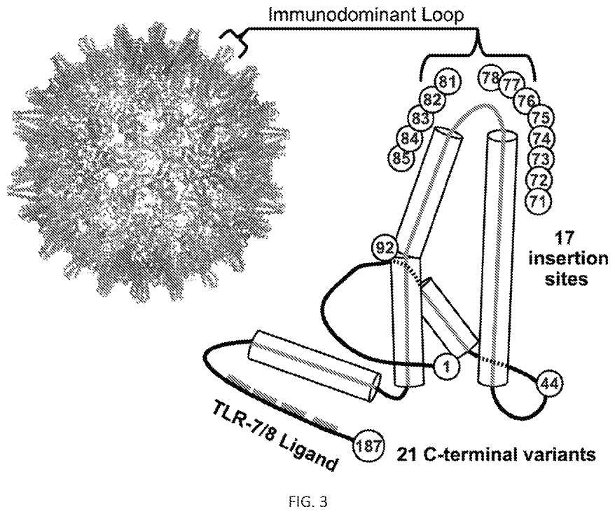

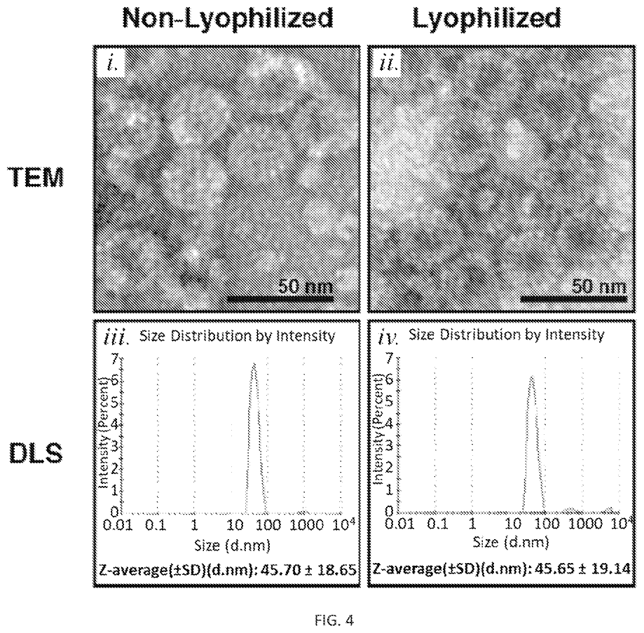

In order to not disrupt self-assembly of the core antigen VLP, the peptide may be inserted inside the loop region for the hepatitis DNA virus core protein sequence, such as, for example, amino acid residues 76, 77, 78, 81, and/or 82 of woodchuck hepatitis DNA virus core antigen, as shown in FIG. 3. In certain embodiments, the insertion into the woodchuck hepatitis DNA virus core antigen may be at position 78 of SEQ ID NO: 6.

The compositions may further comprise excipients or pharmaceutically acceptable carriers. The choice of excipients or pharmaceutically acceptable carriers will depend on factors including, but not limited to, the particular mode of administration, the effect of the excipient on solubility and stability, and the nature of the dosage form.

Excipients and carriers may include any and all solvents, dispersion media, coatings, antibacterial and antifungal agents, isotonic and absorption delaying agents. Some examples of materials which can serve as excipients and/or carriers are sugars including, but not limited to, lactose, glucose and sucrose; starches including, but not limited to, corn starch and potato starch; cellulose and its derivatives including, but not limited to, sodium carboxymethyl cellulose, ethyl cellulose and cellulose acetate; powdered tragacanth; malt; gelatin; talc; excipients including, but not limited to, cocoa butter and suppository waxes; oils including, but not limited to, peanut oil, cottonseed oil, safflower oil, sesame oil, olive oil, corn oil and soybean oil; glycols; including propylene glycol; esters including, but not limited to, ethyl oleate and ethyl laurate; agar; buffering agents including, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants including, but not limited to, sodium lauryl sulfate and magnesium stearate, as well as coloring agents, releasing agents, coating agents, sweetening, flavoring and perfuming agents, preservatives and antioxidants. The compositions of the present invention and methods for their preparation will be readily apparent to those skilled in the art. Techniques and formulations may be found, for example, in Remington's Pharmaceutical Sciences, 19th Edition (Mack Publishing Company, 1995).

The compositions may be formulated for any particular mode of administration including for example, systemic administration (e.g., oral, rectal, nasal, sublingual, buccal, implants, or parenteral) or topical administration (e.g., dermal, pulmonary, nasal, aural, ocular, liposome delivery systems, or iontophoresis).

### 3. Nucleic Acids

The present disclosure provides nucleic acids encoding an antigenic Bacillus anthracis lethal factor peptide. In some embodiments, the antigenic anthrax lethal factor peptide comprises the amino acid sequence of SEQ ID NO: 2 (QIDIRDSLSEEEKELLNRIQ) and/or a functional variant thereof. In certain embodiments, the lethal factor peptide is selected from the group consisting of the amino acid sequences of SEQ ID NO:2, SEQ ID NO:3, SEQ ID NO:4, SEQ ID NO:5, and/or functional variants thereof.

The present disclosure also provides nucleic acids encoding an antigenic Bacillus anthracis protective antigen peptide inserted within an amino acid sequence of a polypeptide capable of forming a virus like particle. In some embodiments, the protective antigen peptide consists of the amino acid sequence of SEQ ID NO: 1 (HGNAEVHASFFDIGGS) and/or a functional variant thereof.

The nucleic acids encoding an antigenic Bacillus anthracis lethal factor peptide and/or an antigenic Bacillus anthracis protective antigen peptide may be inserted within a nucleic acid encoding the amino acid sequence of a polypeptide capable of forming a virus like particle. In some embodiments, the polypeptide capable of forming a virus like particle is a woodchuck hepatitis DNA virus core antigen. In certain embodiments, the amino acid sequence of the woodchuck hepatitis DNA virus core antigen may comprise an amino acid sequence having at least 70% identity (e.g., at 70%, 75%, 80%, 85%, 90%, 95%, 98%, or 99% identity) to SEQ ID NO: 6. In exemplary embodiments, the insertion into the woodchuck hepatitis DNA virus core antigen may be at position 78 of the amino acid sequence SEQ ID NO: 6.

In some embodiments, the polynucleotides disclosed herein can be introduced into an expression vector, such that the expression vector comprises a promoter and the polynucleotides encoding the peptides or polypeptides described herein. The expression vector may allow expression of the peptides or polypeptides in a suitable expression system using techniques well known in the art, followed by isolation or purification of the expressed peptide or polypeptide of interest. A variety of bacterial, yeast, plant, mammalian, and insect expression systems are available in the art and any such expression system can be used. Alternatively, a polynucleotide encoding a peptide of the invention can be translated in a cell-free translation system.

### 4. Vaccines

The immunogenic compositions and immunogens described herein may be used to prepare vaccines.

The vaccine may comprise any of the immunogenic compositions or immunogens described herein and an adjuvant or immunostimulant. Adjuvants and immunostimulants are compounds that either directly or indirectly stimulate the immune system's response to a co-administered antigen. Suitable adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham); mineral salts (for example, aluminum, silica, kaolin, and carbon); aluminum salts such as aluminum hydroxide gel (alum), AlK(SO₄)₂, AlNa(SO₄)₂, AlNH₄(SO₄), and Al(OH)₃; salts of calcium (e.g., Ca₃(PO₄)₂), iron or zinc; an insoluble suspension of acylated tyrosine; acylated sugars; cationically or anionically derivatized polysaccharides; polynucleotides (for example, poly IC and poly AU acids); polyphosphazenes; cyanoacrylates; polymerase-(DL-lactide-co-glycoside); biodegradable microspheres; liposomes; lipid A and its derivatives; monophosphoryl lipid A; wax D from Mycobacterium tuberculosis, as well as substances found in Corynebacterium parvum, Bordetella pertussis, and members of the genus Brucella); bovine serum albumin; diphtheria toxoid; tetanus toxoid; edestin; keyhole-limpet hemocyanin; Pseudomonal Toxin A; choleragenoid; cholera toxin; pertussis toxin; viral proteins; and Quil A. Aminoalkyl glucosamine phosphate compounds can also be used (see, e.g., WO 98/50399, U.S. Pat. No. 6,113,918 (which issued from U.S. Ser. No. 08/853,826), and U.S. Ser. No. 09/074,720). In addition, adjuvants such as cytokines (e.g., GM-CSF or interleukin-2, -7, or -12), interferons, or tumor necrosis factor, may also be used as adjuvants.

Protein and polypeptide adjuvants may be obtained from natural or recombinant sources according to methods well known to those skilled in the art. When obtained from recombinant sources, the adjuvant may comprise a protein fragment comprising at least the immunostimulatory portion of the molecule. Other known immunostimulatory macromolecules which can be used include, but are not limited to, polysaccharides, tRNA, non-metabolizable synthetic polymers such as polyvinylamine, polymethacrylic acid, polyvinylpyrrolidone, mixed polycondensates (with relatively high molecular weight) of 4′,4-diaminodiphenylmethane-3,3′-dicarboxylic acid and 4-nitro-2-aminobenzoic acid (See, Sela, M., Science 166:1365-1374 (1969)) or glycolipids, lipids, or carbohydrates.

Vaccine preparation is a well-developed art and general guidance in the preparation and formulation of vaccines is readily available from any of a variety of sources. One such example is New Trends and Developments in Vaccines, edited by Voller et al., University Park Press, Baltimore, Md., U.S.A. 1978.

The vaccines of the present disclosure may also contain other compounds, which may be biologically active or inactive. For example, one or more immunogenic portions of other antigens may be present, either incorporated into a fusion polypeptide or as a separate compound, within the vaccine. The vaccines may generally be used for prophylactic and therapeutic purposes.

The vaccines may be formulated for any appropriate manner of administration, and thus may be administered by various methods, including for example, topical, oral, nasal, intravenous, intravaginal, epicutaneous, sublingual, intracranial, intradermal, intraperitoneal, subcutaneous, intramuscular administration, or via inhalation.

The vaccines may also comprise buffers (e.g., neutral buffered saline, phosphate buffered saline), carbohydrates (e.g., glucose, mannose, sucrose, dextrans), mannitol, proteins, polypeptides, amino acids such as glycine, antioxidants, bacteriostats, chelating agents such as EDTA or glutathione, solutes that render the formulation isotonic, hypotonic, or weakly hypertonic with the blood of a recipient, suspending agents, thickening agents and/or preservatives. Alternatively, vaccines may be formulated as a lyophilisate. Compounds may also be encapsulated within liposomes using well known technology.

### 5. Methods of Use

The present disclosure provides methods for reducing or preventing anthrax infection in a subject in need thereof. The methods include administering to the subject an effective amount of the immunogenic compositions, immunogens, or vaccines disclosed herein.

An “effective amount” of an antigenic peptide of the invention, and compositions or vaccines thereof, is an amount that is delivered to a subject, either in a single dose or as part of a series, which is effective for inducing an immune response against Bacillus anthracis in the subject. This amount varies depending upon the health and physical condition of the subject to be treated, the capacity of the subject's immune system to synthesize antibodies, the formulation of the peptides, compositions or vaccine, and other relevant factors. It is expected that the amount will fall in a relatively broad range that can be determined by one of skill in the art through routine trials.

The immunogenic compositions, immunogens, or vaccines disclosed herein can be administered in a wide variety of therapeutic dosage forms in the conventional vehicles for topical, oral, systemic, local, and parenteral administration.

The route and regimen of administration will vary depending upon the population and the indication for vaccination and is to be determined by the skilled practitioner. For example, the immunogenic compositions, immunogens, or vaccines disclosed herein may be administered in such dosage forms for example as tablets, capsules (each including timed release and sustained release formulations), pills, powders, granules, elixirs, tinctures, solutions, suspensions, syrups, and emulsions. In some embodiments, the immunogenic compositions, immunogens, or vaccines disclosed herein may be administered by injection. Similarly, they may also be administered parentally, e.g., in intravenously (either by bolus or infusion methods), intraperitoneally, subcutaneously, topically with or without occlusion, or intramuscularly.

The administration may comprise an initial immunization or priming dose and at least one subsequent immunization or booster dose, following known standard immunization protocols. The boosting doses will be adequately spaced at such times where the levels of circulating antibody fall below a desired level. Boosting doses may consist of one or both of the peptides disclosed herein and may comprise alternative carriers and/or adjuvants. The booster dosage levels may be the same or different that those of the initial immunization dosage. Booster doses may be given at, for example, 1 week, 2 weeks, 3 weeks, 4 weeks, two months, three months, 6 months and/or a year later. Alternatively, in some embodiments, the administration may comprise a single immunization (e.g., a single dose of the vaccine).

The specific dose levels may depend upon a variety of factors including the activity of the peptide, composition or vaccine, the age, body weight, general health, and diet of the subject, time of administration, and route of administration. For prophylaxis purposes, the amount of peptide in each dose is an amount which induces an immunoprotective response without significant adverse side effects. The dose range may be established empirically and may range from 10 micrograms to 500 micrograms for the single, priming and/or boosting doses.

The compositions and vaccines may be prepared, packaged, or sold in a form suitable for bolus administration or sold in unit dosage forms, such as in ampules or multi-dose containers containing a preservative.

A second therapy may be used in conjunction with the disclosed immunogens, immunogenic compositions, or vaccines of the present disclosure. The second therapy may be administration of an additional therapeutic agent or an additional vaccine.

### 6. EXAMPLES

### Materials and Methods

Construction of Recombinant Chimeric Woodchuck Hepatitis core antigen virus-like particles The chimeric Woodchuck Hepatitis core antigen (WHcAg) virus-like particles (VLPs) were constructed by modifying a pUC-FLw2 (Full-Length woodchuck) vector expressing the full-length WHcAg protein codon optimized for expression in E. coli essentially as described (Whitacre, et al., 2015 PLOS ONE 10:e0124856, incorporated herein by reference in its entirety). The sequence for FLw2 matches the sequence translated from the woodchuck hepatitis virus core protein open reading frame (accession M18752) and was cloned into a pUC19 vector in place of the multiple cloning site. For inserting heterologous B cell epitopes, EcoRI-Xhol restriction sites were engineered into the FLw2 open reading frame between amino acids 78 and 79 of the core protein gene. The engineered restriction sites add a Gly-Ile-Leu linker on the N-terminal side and a Leu linker on the C-terminal side of the inserted epitopes. Epitopes were cloned into the VLP gene using synthetic oligonucleotides comprising the desired epitope coding sequence and the appropriate engineered restriction sites. All WHcAg constructs were transformed into Alpha-Select competent E. coli (Bioline USA, Inc., Taunton, MA). Following transformation, plasmid DNA was purified by Zymo Zyppy™ Plasmid Miniprep Kit (Zymo Research, Irvine, CA) and correct sequences were confirmed by Sanger DNA sequencing (EuroFins MWG Operon USA, Louisville, KY).

(Woodchuck core protein)-
SEQ ID NO: 6
MDIDPYKEFGSSYQLLNFLPLDFFPDLNALVDTATALYEEELTGRE
HCSPHHTAIRQALVCWDELTKLIAWMSSNITSEQVRTIIVNHVND
TWGLKVRQSLWFHLSCLTFGQHTVQEFLVSFGVWIRTPAPYRPPN
APILSTLPEHTVIRRRGGARASRSPRRRTPSPRRRRSQSPRRRRS
QSPSANC

Purification of VLPs The VLP particles were expressed in Alpha-Select E. coli cells grown in Terrific Broth (Teknova, Hollister, CA). Cells were lysed by passage through an EmulsiFlex-C3 (Avestin, Ottawa, ON, Canada). The lysate was treated with Benzonase® Nuclease (Millipore Sigma, Burlington, MA) and heat denatured for 10 min at 65° C. Lysate was then clarified by centrifugation and then passed through a 0.2 micron filter unit. The WHeAg particles were selectively precipitated by the addition of solid ammonium sulfate to 45% saturation and collected by centrifugation and 10×g. Precipitated VLPs were redissolved in minimum buffer (10 mM Tris, pH 8) and diafiltered with five volume exchanges of final formulation buffer in a hollow fiber cartridge with a 750K molecular weight cutoff (WaterSep BioSeparations, Marlborough, MA). The final formulation buffer is 20 mM Tris, pH8, 100 mM NaCl, 5 mM EDTA, and 5% trehalose.

Endotoxin was removed from the core preparations by phase separation with Triton X-114. Briefly, the VLP solution was made 1% Triton X-114 and incubated at 4° C. for 30 min with mixing, incubated at 37° C. for 10 min, centrifuged at 20,000×g for 10 min at 25° C. and then protein was recovered in the upper phase. This was repeated for a total of 4 extractions. The purified VLPs were 0.2 micron sterile filtered, characterized, and aliquoted. Characterization included custom ELISA, native agarose gel electrophoresis, PAGE, heat stability testing, and optionally circular dichroism and dynamic light scattering. Endotoxin was measured by Pierce™ LAL Chromogenic Endotoxin Quantitation Kit (ThermoFisher Scientific, Waltham, MA).

Animals and Immunizations For generation of rabbit antisera, male and female New Zealand white (NZW) rabbits (distributed equally in each group where possible) (Covance Research Products, Denver PA) were immunized on day 0 with 300 μg of the respective VLPs in Aluminum Hydroxide (Alhydrogel: Superfos, Frederikssund, Denmark) mixed with 100 μg Monophosphoryl Lipid A (Avanti Polar Lipids) in a final volume of 1.0 ml/dose. Rabbits were boosted 5 weeks later with the identical dose of VLP/adjuvant. For assessment of antibody (Ab) responses, rabbits were bled prior to the booster dose at 5 weeks and then 14 and 21 days after the booster dose.

Enzyme-linked immunosorbent assay Antibody responses were assessed by ELISA essentially as previously described (Oscherwitz and Cease, 2015 PLoS ONE 10: e011688, incorporated herein by reference in its entirety). For analysis of antibodies specific for protective antigen (PA) or lethal factor (LF), wells of microtiter plates (Immulon 2, Thermo Labsystems, Franklin MA) were coated overnight at 4° C. with 100 ng of recombinant PA or LF (List Laboratories, Campbell, CA) in a 0.05 M carbonate buffer pH 9.5. Bound Ab was detected with secondary biotinylated Ab specific for rabbit IgG (Southern Biotechnology, Birmingham, AL) followed by streptavidin-alkaline phosphatase and 4-nitrophenylphosphate (Roche, Indianapolis, IN). Absorbance at 405 nm minus absorbance at 650 nm was determined using an ELISA reader (Emax microplate reader, Molecular Devices, Menlo Park, CA). Antibody titers were determined from serial two-fold dilutions of serum and represent the reciprocal dilution at the EC50 established using nonlinear regression to fit a variable slope sigmoidal equation to the serial dilution data using Prism 9.0 (GraphPad Software, Inc., San Diego, CA).

Toxin Neutralization assay (TNA) The ability of Ab to block Lethal toxin (LeTx)-mediated cytotoxicity in vitro was assessed using the RAW264.7 cell line (American Type Culture Collection, Manassas, VA) as described (Oscherwitz et al., 2010 J Immunol 185:3661-3668). For neutralization studies, 13 ng/ml PA (PA83-#171, List labs) was used along with 3 ng of LF (LF-A-#169L, List labs). The reciprocal of the effective dilution protecting 50% of the cells from cytotoxicity (ED50), was determined for each serum by using nonlinear regression to fit a variable slope sigmoidal equation to the serial dilution data set using Prism 9.0. Neutralization data was presented as NF50s which is defined as the EC50 neutralization titer of the test sample/EC50 titer of AVR801. AVR801 (BEI resources, Manassas, VA) is a standardized sample of pooled polyclonal human antisera from individuals immunized with Biothrax. It is used to control for interlaboratory variability in the assessment of toxin neutralization titers through normalization of EC50 titers. The standard TNA assay has a lower limit of detection of 16; titers below this limit were assigned a value of 8. For inhibition studies, rabbit antisera were pre-incubated with 10 uM of inhibitory VLPs for 30 min at RT before being assessed in the TNA. For some analyses, a pooled polyclonal rabbit anti-PA serum, which was produced through immunization of 6 rabbits 5 times at two-week intervals with recombinant PA and complete Freund's adjuvant for priming and incomplete Freund's adjuvant for boosting, is shown for comparison.

Transmission Electron Microscopy and Dynamic Light Scatter Negative stain transmission electron microscopy (TEM) and dynamic light scatter (DLS) were used to evaluate the structure and size distribution of the VLPs, respectively. Where indicated, VLPs were lyophilized according to standard procedures.

### Example 1

### VLP Targeting PA

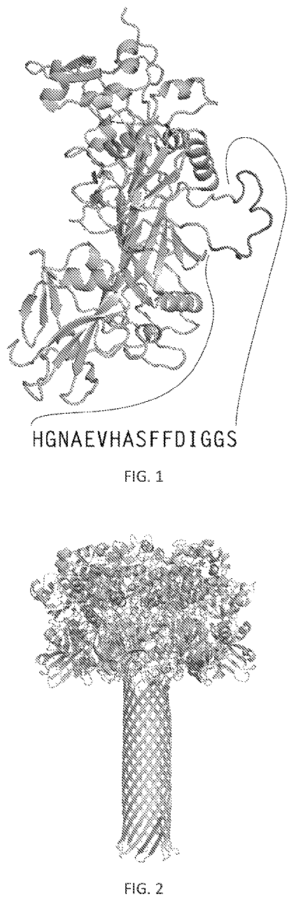

The elicitation of antibodies specific for a linear determinant within the 2B2-283 loop of PA, referred to as the loop neutralizing determinant (LND, FIG. 1), can mediate complete protection of rabbits from an aerosolized spore inhalation challenge with a 200 LD50-targeted dose of B. anthracis Ames strain. The residues within the LND epitope facilitate formation of the beta pore that mediates translocation of the toxins lethal factor and edema factor across cell membranes (FIG. 2). Importantly, an LND-specific antibody is not detectable in Bio Thrax-vaccinee sera. The LND specificity, therefore, is non-overlapping with the specificities elicited by PA, and represents unique specificity for development of a vaccine for anthrax.

VLP vaccines in clinical use include HBsAg vaccines, and the licensed HPV vaccines, most of which are administered with three injections administered over 6 months using aluminum-based adjuvants with Monophosphoryl Lipid A (MPLA). A form of LND vaccine was developed and optimized which displays the LND neutralizing epitope on virus like particles (VLPs) (FIG. 3). One example of LND-VLP, has the anthrax LND sequence (SEQ ID NO: 1 HGNAEVHASFFDIGGS) inserted within the hepatitis capsid major immunodominant region at position 78. Data in rabbits unequivocally demonstrated that this LND-VLP vaccine has the potential to elicit potent and rapid protective immunity against anthrax with only one or two injections in human use adjuvants, was lyophilizible without loss of immunogenicity, and could be uniquely valuable for use against potential reengineered strains of B. anthracis, and in imminent pre-exposure and postexposure scenarios.

To assess what effect lyophilization might have on the physical characteristics and immunogenicity of the lead LND-VLP (VLP66), VLP66 was lyophilized, stored the material at room temperature for several weeks and the resuspended the nanoparticle in sterile water. This material is hereafter referred to as VLP66-LYO. FIG. 4 is the transmission electron microscopy (TEM) of the soluble VLP66 (panel i) which had not undergone lyophilization, and the lyophilized and resuspended VLP66-LYO (panel ii), and demonstrated that both form approximately 40-50 nanometer, icosahedral particles. Panels ili and iv, show the size distributions of the VLP66 and VLP66-LYO, respectively, as determined by dynamic light scatter (DLS), and demonstrated that both populations of particles have similar size distributions and nearly identical average particle diameters of 45.7 and 45.65 nanometers for the VLP66 and VLP66-LYO, respectively.

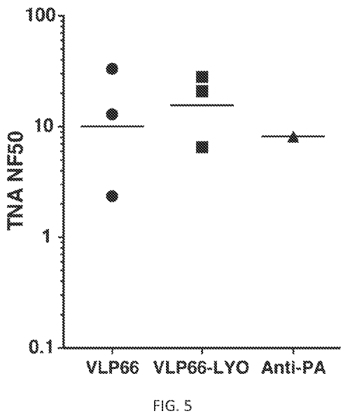

To compare the immunogenicity of VLP66 and VLP66-LYO, groups of rabbits (n=3) were immunized with the respective VLPs s.c./i.m. with 300 μg priming doses and 300 μg boosting doses employing Alhydrogel mixed with 100 μg of Monophosphoryl Lipid A (MPLA) in a total volume of 1.0 ml per rabbit per dose on day 0 and at 5 weeks, respectively (hereafter referred to as the “standard protocol”). As shown in FIG. 5, immunization with both the VLP66 or the VLP66-LYO elicited exceedingly high and protective levels of neutralizing Ab at the 8-week time point (3 weeks post-boost).

Prior work demonstrated that an NF50≥0.56 had a 70% probability of protecting rabbits from B. anthracis Ames strain spore challenge and a NF50 of 1.68, a 97% probability of survival from spore challenge. All rabbits in the VLP66 and VLP66-LYO groups, therefore, would likely survive a lethal anthrax spore challenge. A pooled polyclonal rabbit anti-PA serum which was produced through immunization of 6 rabbits 5 times at two-week intervals with recombinant PA using complete Freund's adjuvant for priming and incomplete Freund's adjuvant for boosting is shown for comparison.

The optimized LND-VLP was highly immunogenic in rabbits using human use adjuvants and elicited exceedingly high specific activities and protective levels of neutralizing Ab with two immunizations. The lyophilized and resolubilized LND-VLP vaccine had a particle morphology akin to non-lyophilized vaccine and did not demonstrate any loss of immunogenicity compared to the LND-VLP that had not undergone lyophilization.

### Example 2

### Lethal Factor Neutralizing Epitope and Vaccines

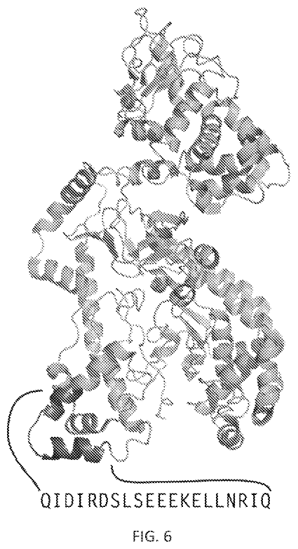

A novel linear neutralizing epitope in lethal factor (LF) from B. anthracis was identified. An antibody to this neutralizing specificity was nonoverlapping with antibody elicited to BioThrax. Using this epitope, a new form of LF vaccine was developed and optimized which displays the LF neutralizing epitope (FIG. 6) on VLPs (FIG. 3). Data in rabbits demonstrated that this LF-VLP vaccine has the potential to elicit potent and rapid protective immunity against anthrax with only one or two injections in human use adjuvants, is lyophilizable without loss of immunogenicity, and could be uniquely valuable for use against potential reengineered strains of B. anthracis, and in imminent pre-exposure and post-exposure scenarios.

A panel of LF-VLPs (Table 1) was used to immunize groups of rabbits (n=3) s.c./i.m. with 300 μg priming doses and 300 μg boosting doses employing Alhydrogel/MPLA on day 0 and at 5 weeks, respectively, as described above.

TABLE 1
LF target epitope sequence inserted into
the woodchuck VLP major immunodominant
region at position 78.

| Construct (PDB | |
|---|---|
| numbering) | Amino Acid Sequence |
| VLP147 (332-361) | TEEKEFLKKLQIDIRDSLS |
| | EEEKELLNRIQVDSSN |
| | (SEQ ID NO: 3) |
| VLP244 (338-361) | LKKLQIDIRDSLSEEEKEL |
| | LNRIQVDSSN |
| | (SEQ ID NO: 4) |
| VLP237 (341-366) | QIDIRDSLSEEEKELLNR |
| | IQVDSSN |
| | (SEQ ID NO: 5) |
| VLP148 (341-361) | QIDIRDSLSEEEKELLNR |
| | IQ (SEQ ID NO: 2) |

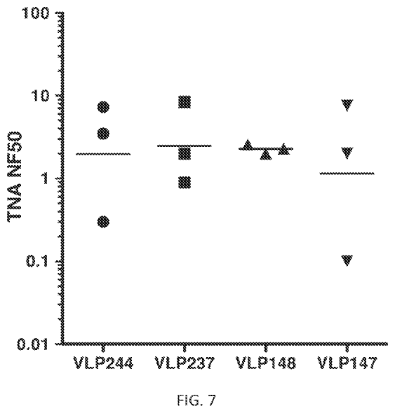

All constructs elicited high levels of toxin-neutralizing antibody thereby validating this site and the potential of these constructs for use as vaccines designed to protect from anthrax. Moreover, the results defined segments 20 to 35 amino acids in length that could elicit this response when displayed on the LF-VLP. However, none of the groups with variations in the LF peptide target sequence demonstrated significantly superior geometric mean neutralization titers by ANOVA (FIG. 7). VLP148 was the shortest peptide sequence evaluated and contains the minimal neutralizing target epitope among the peptide sequences evaluated. VLP148 and VLP237 were most consistent in the elicitation of neutralizing Ab and all rabbits in these groups would be predicted to survive anthrax spore challenge at the 8-week time point based on surrogate neutralization data in rabbits. Serum from several rabbits possessed extremely high levels of neutralizing Ab, highlighting the potential of this epitope as a protective target.

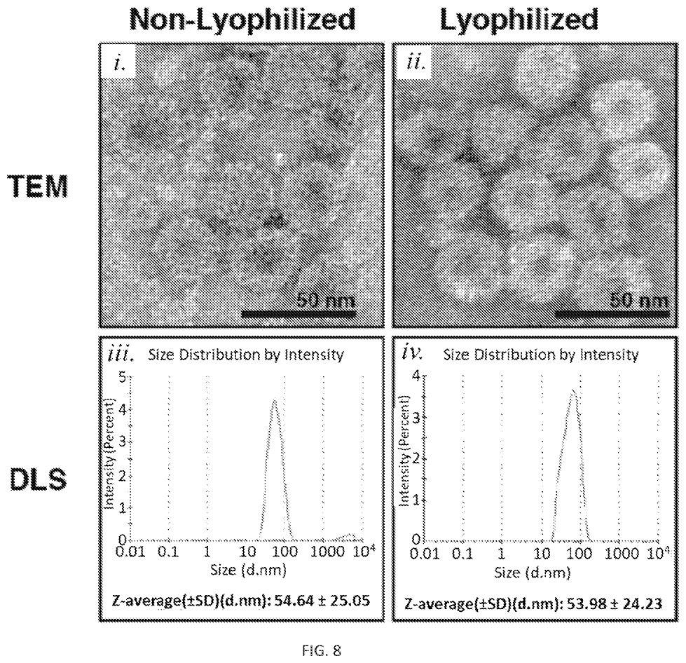

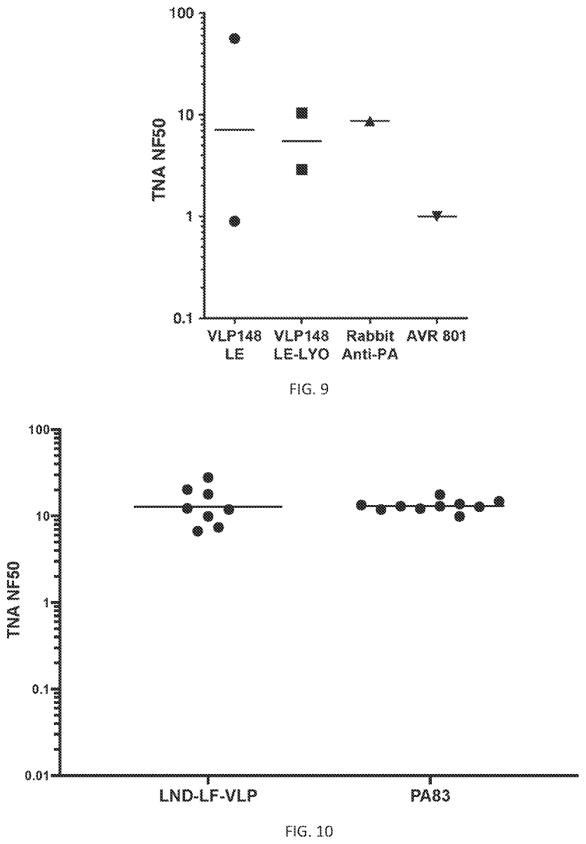

Prior to lyophilization of the VLP148, a laboratory-scale technique was employed to remove endotoxin using extraction with Triton X114. Using the Limulus amebocyte lysate assay (LAL), extraction with Triton X114 was found to reduce endotoxin levels of the VLP148 from 90,647 EU/mg in the untreated sample to 16 EU/mg in the Triton-extracted sample, hereafter referred to as VLP148 Low Endotoxin or VLP148LE. It has been previously shown in mice that the presence of bacterial endotoxin does not affect the immunogenicity of the VLP vaccines, nevertheless it was important to demonstrate experimentally that endotoxin was not contributing to the immunogenicity of the VLPs. VLP148LE (low endotoxin) was lyophilized (VLP148LE-LYO), and following 3 weeks of storage at RT, VLP148LE-LYO was resolubilized and both VLPs were evaluated by TEM and DLS. Similar to the results observed with the LND-VLP, there was no detectable effect on either the physical characteristics or size distribution of the VLP particles that could be attributable to lyophilization (FIG. 8, panels i-iv). To compare the respective immunogenicities of the VLP148LE and VLP148LE-LYO, as well as to evaluate whether the low endotoxin VLPs retain immunogenicity profiles comparable to the VLP148 prior to endotoxin reduction, the immunogenicity was compared using Alhydrogel/MPLA and the standard protocol in groups of rabbits (n=2) immunized with either the VLP148LE or the VLP148LE-LYO. Rabbits immunized with the VLP148LE-LYO elicited protective levels of LF-specific neutralizing Ab after two immunizations and the lyophilization did not lead to any significant diminution of immunogenicity compared to the group of rabbits immunized with the non-lyophilized VLP148LE (FIG. 9). Comparison of the TNA titers in both low endotoxin groups (VLP148LE and VLP148LE-LYO) with the VLP148 (FIG. 7) suggested that the endotoxin does not contribute to immunogenicity of the VLPs in rabbits.

The LF-VLP was highly immunogenic and elicited levels of neutralizing Ab which would be predicted to protect rabbits from aerosol spore challenge. The lyophilized and resolubilized low endotoxin LF-VLP vaccine had a particle morphology and size distribution akin to non-lyophilized vaccine and did not demonstrate any loss of immunogenicity compared to non-lyophilized LF-VLP. In addition, both the low endotoxin VLPs, VLP148LE-LYO and VLP148LE, did not show any reduction in immunogenicity in rabbits compared to the VLP148 which contained significantly more endotoxin, suggesting that endotoxin does not contribute to the immunogenicity of the VLPs in this species.

### Example 3

### Combination PA and LF

The immunogenicity of a combined mixture of the LND-VLP/LF-VLP was evaluated in rabbits. Both the LND-VLP (VLP66) and LF-VLP (VLP148) were lyophilized and resolubilized before being used to immunize a group of rabbits (n=8) with the combined LND/VLP and LF/VLP. Each rabbit was given the standard dose of each VLP, 300 μg of the LND-VLP and 300 μg of the LF-VLP mixed with 100 μg of MPLA in Alhydrogel in a total volume of 1.0 ml per rabbit per dose. Rabbits were boosted with the identical immunogen/adjuvant dose at 5 weeks (the standard protocol). For comparison to the gold standard in this model, a second group of rabbits (n=10) was immunized with 50 μg of PA83 in Alhydrogel at day 0 and 28 according to established protocols (Little et al., 2006). All rabbits were bled at week 8 for assessment of Ab responses. As shown in FIG. 10, sera obtained from rabbits immunized with the combined, bivalent LND-VLP/LF-VLP at the 8 week time point demonstrated extremely high and protective levels of neutralizing antibody. The responses were almost indistinguishable in group-specific geometric mean titer (GMT) expressed as NF50s to the neutralizing Ab responses in the sera from rabbits immunized with PA83, with GMTs of 12.81 and 13.12, respectively.

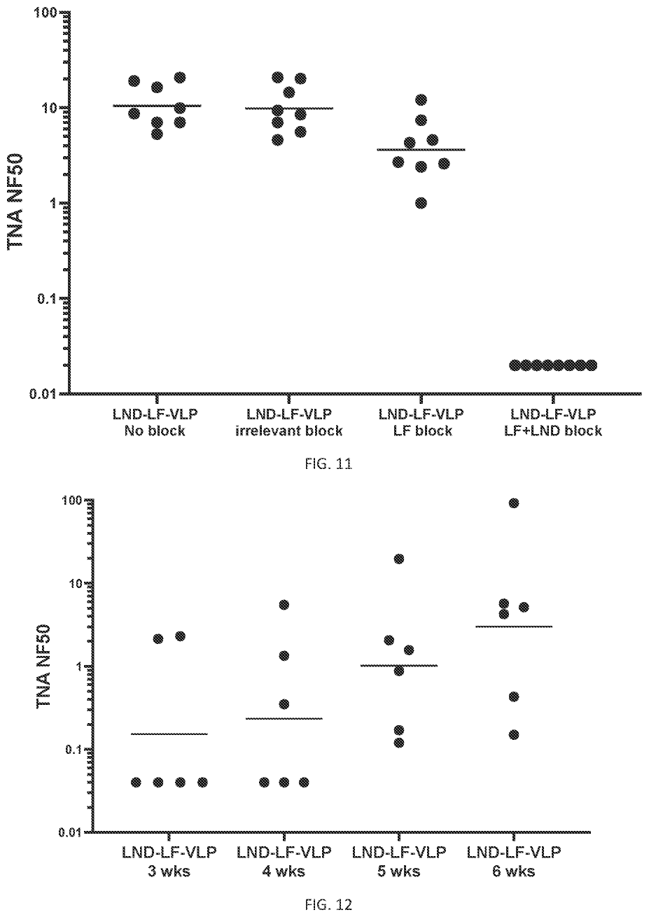

To determine the distribution of neutralizing Ab specificities in the sera from rabbits immunized with the LND-VLP/LF-VLP vaccine, inhibition experiments were performed to delineate the respective contributions to neutralization associated with LND- and LF-specific antibodies. For the neutralization inhibition assay, individual 8 week rabbit serum samples were pre-incubated with either an irrelevant VLP (irrelevant block), LF-VLP (LF block), both LF- and LND-VLPs (LF+LND block), or with media alone (no block). As shown in FIG. 11, pre-incubation with either media alone or an irrelevant VLP gave similar results, with geometric mean neutralization titers of 10.5 and 9.8, respectively. By contrast, pre-incubation with the LF-VLP led to an approximate 64% reduction in the geometric mean neutralization compared to the geometric mean titer in the irrelevantly blocked group, suggesting that in aggregate, the LF-specific neutralization represented 64% of the neutralization in the bivalent, LND-VLP/LF-VLP-immunized rabbits, while the LND-specific neutralization represented 36% of the neutralizing antibody in this cohort. As also shown, pre-incubation of the rabbit sera with both the LND-VLP and the LF-VLP led to complete abrogation of the neutralization.

The immunogenicity of the LND-VLP/LF-VLP vaccine in rabbits following only a single immunization using a water-in-oil emulsion was also evaluated. Each rabbit was given the standard dose of each VLP, 300 μg of the LND-VLP and 300 μg of the LF-VLP in a final volume of 0.5 mls mixed 1:1 by volume with 0.5 mls of Incomplete Freund's adjuvant. Rabbits were injected a single time with 1.0 ml of the emulsion subcutaneously at day 0 and were then bled at 3, 4, 5 and 6 weeks for assessment of the antibody and neutralization titers. As shown in FIG. 12, at 3 weeks post-immunization, two out of six rabbits had levels of neutralization previously demonstrated to predict a high probability of protection from B. anthracis Ames strain aerosol spore challenge (NF50 of 0.56 predicts a 70% or 88% probability of survival in rabbits and non-human primates respectively; Ionin B et al, 2013, CVI 20:1016) and by week 6, five out of 6 rabbits would be predicted to survive the spore challenge based on their levels of serum neutralization activity.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art and may be made without departing from the spirit and scope thereof.

What is claimed is:

1. An immunogenic composition comprising at least one or each of:
an antigenic Bacillus anthracis lethal factor peptide; and
an antigenic Bacillus anthracis protective antigen peptide,
wherein when the lethal factor peptide is absent the protective antigen peptide is inserted within an amino acid sequence of a polypeptide capable of forming a virus like particle.

2. The immunogenic composition of claim 1, wherein the protective antigen peptide comprises the amino acid sequence of SEQ ID NO: 1.

3. The immunogenic composition of claim 1 or 2, wherein the lethal factor peptide comprises the amino acid sequence of SEQ ID NO: 2.

4. The immunogenic composition of any of claims 1-3, wherein the lethal factor peptide is selected from the group consisting of the amino acid sequences of SEQ ID NO:2, SEQ ID NO:3, SEQ ID NO: 4, and SEQ ID NO:5.

5. The immunogenic composition of any of claims 1-4, wherein the lethal factor peptide is inserted within an amino acid sequence of a polypeptide capable of forming a virus like particle.

6. The immunogenic composition of any of claims 1-5, wherein when the lethal factor peptide is present, the protective antigen peptide is inserted within an amino acid sequence of a polypeptide capable of forming a virus like particle.

7. The immunogenic composition of claim 6, wherein the lethal factor peptide and the protective antigen peptide are each individually inserted within an amino acid sequence of a polypeptide capable of forming a virus like particle.

8. The immunogenic composition of any of claims 1-7, wherein the polypeptide capable of forming a virus like particle is a woodchuck hepatitis DNA virus core antigen.

9. The immunogenic composition of claim 8, wherein the amino acid sequence of the woodchuck hepatitis DNA virus core antigen comprises an amino acid sequence having at least 70% identity to SEQ ID NO: 6.

10. The immunogenic composition of claim 9, wherein insertion in the amino acid sequence of the woodchuck hepatitis DNA virus core antigen is at position 78 of SEQ ID NO: 6.

11. The immunogenic composition of any of claims 7-10, wherein the lethal factor peptide and the protective antigen peptide are in distinct virus like particles.

12. A vaccine comprising the composition of any of claims 1-11 and at least one adjuvant.

13. A method for reducing or preventing anthrax infection in a subject in need thereof, comprising administering to the subject an effective amount of the composition of any of claims 1-11 or the vaccine of claim 12.

14. The method of claim 13, wherein the administering comprises an initial immunization and at least one subsequent immunization.

15. The method of claim 13, wherein the administering comprises a single immunization.

16. Use of the immunogenic composition of any of claims 1-11 in the manufacture of a medicament for the treatment or prevention of anthrax.

17. A nucleic acid encoding an antigenic Bacillus anthracis lethal factor peptide.

18. The nucleic acid of claim 17, wherein the lethal factor peptide comprises the amino acid sequence of SEQ ID NO: 2.

19. The nucleic acid of claim 17 or 18, wherein the antigenic anthrax lethal factor peptide is selected from the group consisting of the amino acid sequences of SEQ ID NO:2, SEQ ID NO:3, SEQ ID NO: 4, and SEQ ID NO:5.

20. The nucleic acid of any of claims 17-19, wherein the nucleic acid is inserted within a nucleic acid encoding the amino acid sequence of a polypeptide capable of forming a virus like particle.

21. The nucleic acid of claim 20, wherein the polypeptide capable of forming a virus like particle is a woodchuck hepatitis DNA virus core antigen.

22. The nucleic acid of claim 21, wherein amino acid sequence of the woodchuck hepatitis DNA virus core antigen comprises an amino acid sequence having at least 70% identity to SEQ ID NO: 6.

23. An expression vector comprising the nucleic acid of any of claims 17-22 in combination with a promoter.

24. A nucleic acid encoding an antigenic Bacillus anthracis protective antigen peptide inserted within an amino acid sequence of a polypeptide capable of forming a virus like particle.

25. The nucleic acid of claim 24, wherein the protective antigen peptide consists of the amino acid sequence of SEQ ID NO: 1.

26. The nucleic acid of claim 24 or 25, wherein the polypeptide capable of forming a virus like particle is a woodchuck hepatitis DNA virus core antigen.

27. The nucleic acid of claim 26, wherein amino acid sequence of the woodchuck hepatitis DNA virus core antigen comprises an amino acid sequence having at least 70% identity to SEQ ID NO: 6.

28. An expression vector comprising the nucleic acid of claim 27 in combination with a promoter.
